(19) United States
(12) Patent Application Publication
(10) Pub. No.: US 2010/0106135 A1
(43) Pub. Date: Apr. 29, 2010

(54) ANGIOCATHETER AND METHOD OF OPERATING THE SAME

(76) Inventor: Reza Radmand, Encino, CA (US)

Correspondence Address: Law Offices of Daniel L. Dawes;Dawes Patent Law Group, 5200 Warner Blvd, Ste. 106, Huntington Beach, CA, 92649, US

(21) Appl. No.: 12/260,622

(22) Filed: Oct. 29, 2008

(51) Int. Cl. A61M 25/06

(52) U.S. Cl. 604/508; 604/164.01

(57) ABSTRACT

An improved angiocatheter device for introducing intravenous fluids or drugs into the lumen of a patient through a sheath. At the point of the procedure where a needle needs to be removed but the sheath is to remain implanted, a spring in the needle portion of the device is expanded thereby retracting the needle and allowing the operator to remove the needle from the sheath and an introducer hub. This feature allows for quick insertion and removal from the patient without the necessity of sliding or otherwise removing the needle from the sheath in a multiple step process. In this manner, the catheter or lead can implanted quickly and safely while also protecting the operator from accidental exposure to the patient's blood and eliminating the need to apply positive pressure to the lumen while the needle is being removed.

### BACKGROUND OF THE INVENTION

1. Field of the Invention

The invention relates to the field of angiocatheters, in particular the insertion of catheters and leads into the lumens of a patient's body and in particular, into the vascular system.

2. Description of the Prior Art

Inserting a catheter or other medical lead for the purposes of connecting a patient to an intravenous line or other medical device has long been known in the prior art. Typically, a needle or other puncture device is inserted into the lumen or vessel of a patient. Once the lumen wall has been breached, a catheter or similar type medical lead slides over the needle, and then the needle is withdrawn leaving the medical catheter in its place. While the needle is being withdrawn from the patient however, outside pressure must be applied along the lumen in order to prevent blood from accidentally spilling out of the proximal end of the catheter and possibly exposing the medical staff to the blood contaminants. This outside pressure must be applied therefore until an intravenous line or other medical device is coupled to the proximal end of the catheter and the pressure differential between the patient and the outside environment is eliminated.

Many techniques and devices found in the prior art have attempted to simplify the catheter insertion process, however even if they can prevent blood from spilling out of the proximal end of the catheter, they all still require at least two steps to complete the procedure. For example, after the catheter or lead has been successfully inserted, the valve or introducer portion of the device must then be slid over the catheter or otherwise removed from the operation area. This extra step is unnecessary and can even add some discomfort to the patient as the introducer is maneuvered away as well as increase the length of the procedure.

What is needed is a device that not only prevents the accidental spilling of blood from the inserted catheter and eliminates the need for applying pressure to the lumen while an intravenous line or other medical device is attached, but also makes the entire catheter insertion procedure a one-step process.

### BRIEF SUMMARY OF THE INVENTION

The illustrated embodiment of the invention is an apparatus for inserting a medical lead into the lumen or vessel of a patient comprising a introducer assembly and a needle assembly, wherein the needle assembly is wholly removable from the introducer assembly.

The introducer assembly further comprises a central hub, a sheath coupled to the distal end of the central hub, a cap coupled to the proximal end of the central hub, and a medication or fluid line coupled to the central hub.

The needle assembly further comprises a housing, a moveable base disposed inside of the housing, a spring disposed between the moveable base and the distal end of the housing, and a needle coupled to the moveable base and threaded through the center of the spring.

The cap coupled to the distal end of the central hub is comprised of a material that allows it to be self-sealing each time it is punctured by an outside medical puncturing device, including the needle that is coupled to the moveable base within the needle assembly.

The medication line that is coupled to the central hub is in turn coupled to an outside medical supply device, such as an intravenous bag or medicament supply.

The needle in the needle assembly is long enough to transverse through the inside of the introducer assembly, extend into the body of the patient and puncture a lumen or vessel wall of the patient.

Once the needle has breached or punctured the lumen or vessel wall, the sheath coupled to the distal end of the central hub is slid distally over the needle.

It is further an aspect of the illustrated embodiment of the invention that the needle assembly comprises a means for separating from the introducer assembly, preferably with a means for retracting the needle from the inside of the introducer assembly and withdrawing entirely into the housing of the needle assembly.

The present application also includes a method of inserting a medical lead into the lumen or vessel of a patient comprising placing a introducer assembly over the lumen or vessel of the patient where a medical lead is to be inserted, driving a needle from a needle assembly through the introducer assembly into the patient, puncturing the lumen or vessel wall of the patient, sliding the distal portion of the medical lead coupled to the distal end of the introducer assembly over the needle and into the lumen of the patient, withdrawing the needle from the lumen of the patient and introducer assembly, and separating the needle assembly from the introducer assembly.

In one embodiment of the invention, the method of driving a needle from a needle assembly through the introducer assembly preferably comprises compressing a spring disposed in the needle assembly and puncturing a self-sealing cap disposed on the proximal end of the introducer assembly.

In another embodiment of the invention, the method of withdrawing the needle from the lumen of the patient and the introducer assembly preferably comprises retracting the needle into a housing of the needle assembly via the movement of an expanding spring disposed in the needle assembly and retracting the needle through a self-sealing cap disposed on the proximal end of the introducer assembly.

In yet another embodiment of the invention, the method of separating the needle assembly from the introducer assembly preferably comprises retracting the needle into a housing of the needle assembly via the movement of an expanding spring disposed in the needle assembly and retracting the needle through a self-sealing cap disposed on the proximal end of the introducer assembly.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side plan view of the angiocatheter device as it is being inserted into the body of a patient.

FIG. 2 is a side plan view of the angiocatheter device once it has breached the lumen wall of the patient.

FIG. 3 is a side plan view of the angiocatheter device after the needle assembly has been retracted from the introducer assembly of the device leaving the introducer assembly of the device inside the lumen of the patient.

FIG. 4 is a plan view of the entire angiocatheter device attached to an intravenous line, but when it is not yet partially implanted into the body of a patient.

FIG. 5 is a cut away side view of the angiocatheter device when the needle assembly is in the contracted position and the needle is extended into the introducer assembly of the device by compressing the internal spring in the needle assembly.

FIG. 6 is a cut away side view of the angiocatheter device when the needle assembly is being withdrawn from the introducer assembly of the device by relaxing the internal spring in the needle assembly.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

An improved angiocatheter device for introducing a catheter or other medical lead into the lumen or vessel of a patient is disclosed in the following. In the following reference will be made generally to a lumen, but it is to be understood that a lumen includes any body cavity and in particular any vessel or artery in the vascular system. At the point of the medical procedure where an insertion means or needle assembly is to be removed with the catheter or lead remaining implanted, a spring in the needle portion of the device is expanded thereby retracting a needle from the puncture site and allowing the operator to remove the needle portion from the device. This feature allows for quick insertion and removal from the patient without the necessity of sliding or otherwise removing the insertion means or needle assembly from the catheter in a multiple step process. In this manner, the catheter or lead can implanted quickly and safely while also protecting the operator from accidental blood splatter or other blood exposure to the patient and eliminating the need to apply positive pressure to the lumen or vessel while the insertion mean or needle assembly is being removed.

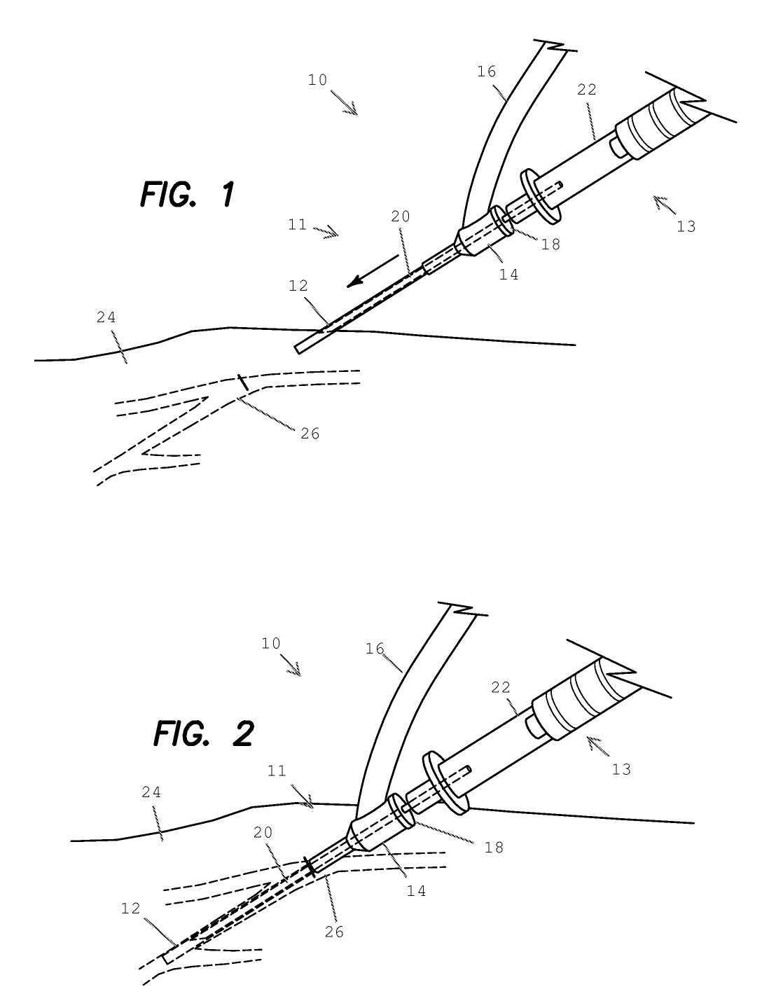

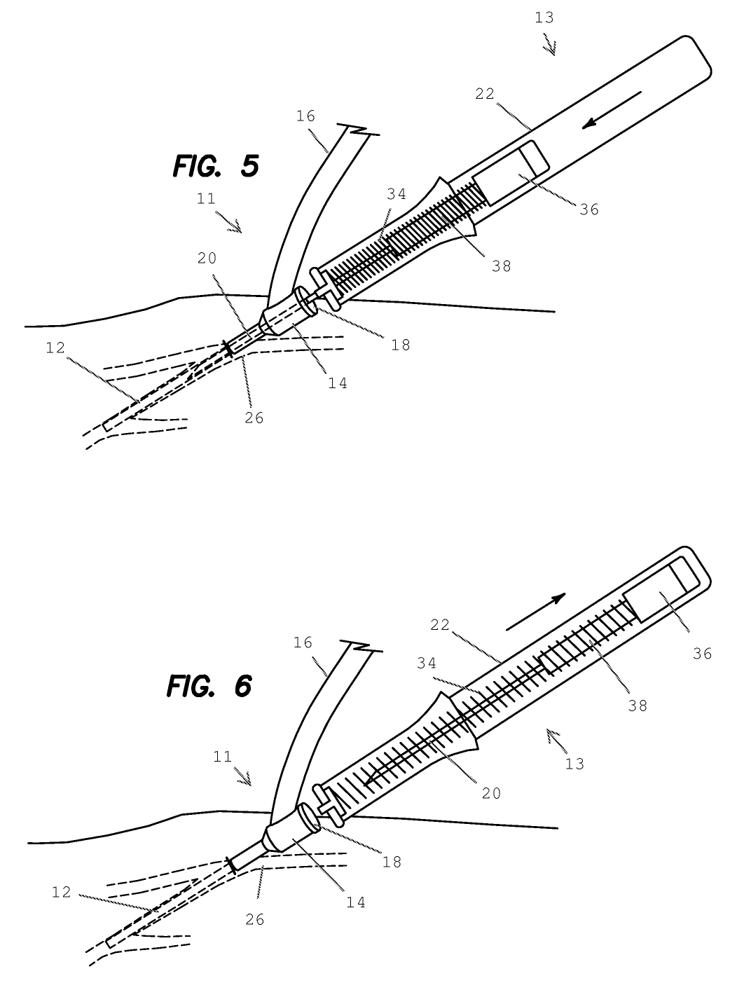

A side plan view of an angiocatheter device is shown in FIG. 1 and is generally noted by reference numeral 10. The angiocatheter 10 comprises an introducer assembly 11 that is best seen in FIGS. 1 and 2 and a needle assembly 13 as best seen in FIGS. 5 and 6.

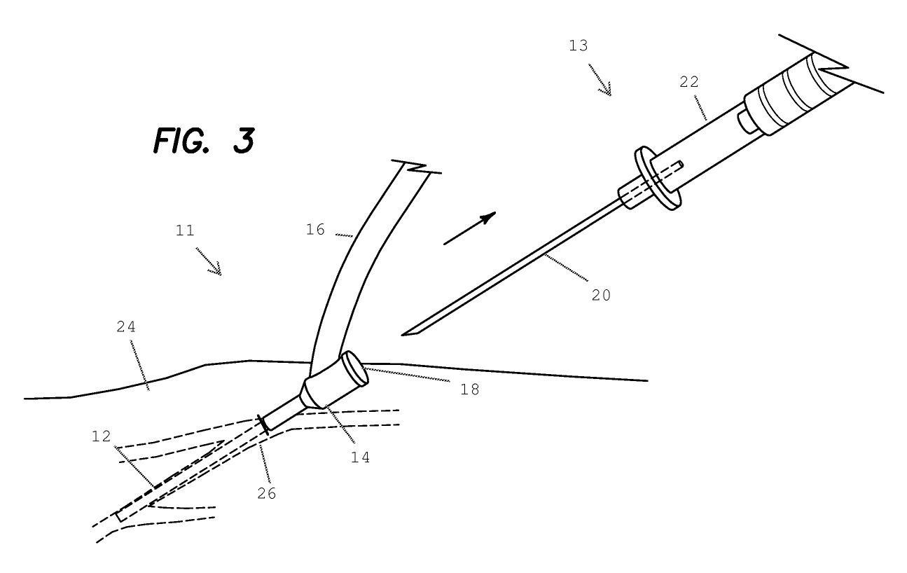

The introducer assembly 11 of the device shown in FIGS. 1 and 2 comprises an introducer hub 14 that in the illustrated embodiment is substantially cylindrical in shape and tapers down to a smaller diameter at its distal tip than at its proximal end. The introducer hub 14 is made out of a sterile flexible Teflon material that is well known to those in the art. At the distal tip of the introducer hub 14 is an introducer sheath 12. The introducer sheath 12 is the portion of the device that remains in the lumen of a patient and is made out of the same flexible Teflon material as the introducer hub 14. Coupled to the side of the introducer hub 14 is an side port and line 16 which is in turn coupled to an intravenous (IV) hub 28 as depicted in FIG. 3 or other drug and/or fluid supply. The line 16 receives IV fluid from the IV hub 28 and directs it into the introducer hub 14. At the proximal end of the introducer hub 14 is a cap 18. The cap 18 is made from the same rubber material, valve or self-sealing membrane that is common to IV lines which allows an operator to administer medications by puncturing the introducer cap 18 as many times as needed with a medical needle insertion tool such as a syringe without the possibility of any fluid leaking back out of the hub 14 once the medication has been administered.

Turning to FIGS. 5 and 6, the angiocatheter device 10 also comprises a needle assembly 13. The needle assembly 13 comprises a housing 22 that may be made out of plastic or any composite known in the art. A base 36 that is cylindrical in shape rests in the inside of the housing 22 and has a outer radius that is slightly smaller than that of the inner radius of the housing 22 allowing it to freely slide both distally and proximally within the housing 22 while maintaining a tight or secure sliding fit with the housing 22. Base 36 may have a tab (not shown) extending through a longitudinal slot (not shown) in housing 22, or may be provided with a plunger or piston (not shown) similar to syringe piston which freely slides in housing 22 to allow it to be manually driven toward the distal end of housing 22 and later released. In one embodiment, the dimensions of the elements of the illustrated embodiment are such that the index finger of the hand of the operator holding housing 22 can be inserted into the proximal end of housing 22 used for driving base 36. Alternatively, there may be provided a pneumatic, electric or mechanical mechanism of conventional design for driving base 36 toward the distal end of housing 22 and then selectively releasing it, which mechanism can be controlled by a foot switch or manual switch mounted on needle assembly 22 or other convenient location. Coupled to the distal end of base 36 is a core 38 that is made of the same material as base 36. A compression spring 34 is telescopically disposed over core 38 and its proximal end bears against the distal end of base 36. The core 38 has a smaller diameter than the base 36 and slides with the base 36 until the proximal end of the core 38 reaches or nears the distal end of the housing 22 at which point it is stopped by contact with the distal end of the bore defined in housing 22, by the maximal possible compression of spring 34 or other conventional means, thus preventing the distal end base 36 from ever reaching the most distal point of the housing 22 and providing a limit stop or controlled disposition of needle 20 from housing 22.

A needle 20 shown by perforated lines in FIGS. 1, 2, and 5 is in turn coupled to the core 38. The needle 20 has a diameter to effectively pierce the lumen 26 of a patient so as to provide a sufficient opening for introducer sheath 12 to enter the lumen 26 thereafter through the puncture site. The needle 20 is also of sufficient length so that when the needle assembly 13 is in the compressed or contracted position as best seen in FIG. 5, the needle extends out of the housing 22 and through the introducer assembly 11 including introducer sheath 12 when spring 34 is compressed to a sufficient degree (not shown). The housing 22 is in turn long enough so that when the needle assembly 13 is in the expanded position as best seen in FIG. 6, the needle 20 is completely withdrawn into and enclosed within the housing 22.

The needle assembly 13 includes spring 34 as best seen in FIG. 5. The spring 34 is disposed around the core 38 between the base 36 and the distal end of the housing 22. The spring has a sufficient outer diameter that is larger than the outer diameter of core 38 but is smaller than the inner diameter of the housing 22.

Returning to FIG. 1, when the angiocatheter device 10 is to be used to insert a catheter or other medical lead in a patient 24, the angiocatheter 10 is positioned at a puncture site 25 above the patient's lumen 26 that is to be entered. As seen in FIG. 5, the base 36 is then pushed distally within the housing 22 of the needle assembly 13 either by manual or mechanical means which in turn pushes the core 38 and needle 20 distally towards the introducer cap 18 and compresses the spring 34. As the needle 20 is pushed out of the housing 22 and spring 34 compressed, needle 20 punctures the introducer cap 18 and continues through the hollow introducer hub 14 and introducer sheath 12 until it makes contact with the patient 24 at the puncture site 25. The needle 20 is then inserted into the patient 24 at the puncture site 25 until it punctures or breeches the wall of the lumen 26. At this point, the distal movement of the needle 20 is stopped by the operator and the introducer sheath 12 is then slid distally over the needle 20 and into the lumen 26 as seen in FIGS. 2 and 5 assisted in part by the expansion of spring 34.

Once the introducer sheath 12 has been placed inside the lumen 26 at the proper position, the operator releases the distally-directed pressure placed on the base 36 via manual or mechanical means and spring 34 then freely expands against the base 36, driving it, the core 38 and the needle 20 in the proximal direction. The needle 20 in turn is then withdrawn back through the introducer sheath 12, through the introducer hub 14 and out of the introducer cap 18 and into the housing 22 as seen in FIG. 6. As the needle 20 exits the introducer assembly 11 portion of the angiocatheter 10 through the introducer cap 18, the cap 18 seals itself thus preventing any of the patient's blood from splattering or escaping the device 10 and coming into contact with the operator. With the needle 20 completely removed from the introducer cap 18, the needle assembly 13 is effectively removed from the introducer portion of the device 10 as seen in FIG. 6, leaving the introducer sheath 12 within the patient 24 and the introducer hub 14 and introducer line 16 outside of the patient 24. The needle is also completely encapsulated within housing 22 thereby preventing accidental sticks or punctures from needle 20 as the needle assembly 13 is handled thereafter by the operator.

Alternatively, after the introducer sheath 12 has been placed in the lumen 26, the operator may remove the needle assembly 13 from the introducer assembly 11 by first extracting the needle 20 from the introducer cap 18 while the spring 34 is still in the compressed position as seen in FIG. 3. After the needle 20 has cleared the introducer cap 18, the cap 18 seals itself and the operator releases the distal pressure on the base 36 thus drawing the needle 20 within the housing 22 as described above. Manual withdrawal of needle 20 from assembly 11 and spring driven withdrawal of needle into housing 22 may of course occur simultaneously depending on operator manipulation.

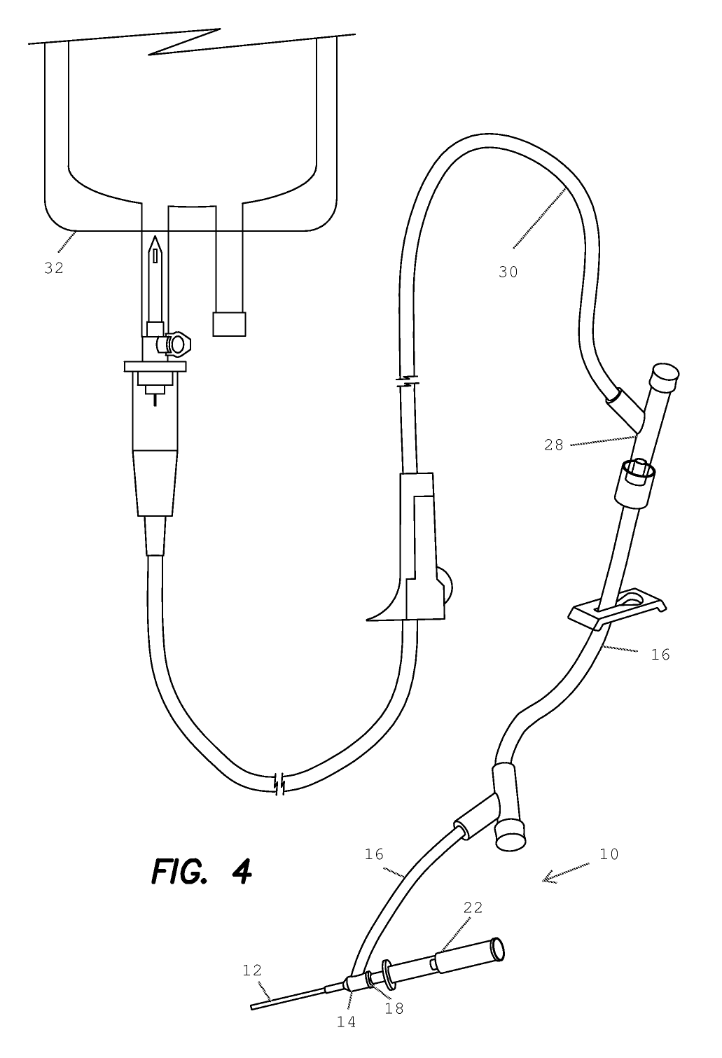

The line 16 that is coupled to the introducer hub 14 may be connected to any number of medical devices such as an intravenous (IV) bag 32 as shown in FIG. 4. The line 16 is sufficiently long enough so that it may provide an effective conduit from the introducer hub 14 to an IV connector 28. The IV connector 28 is then in turn coupled to an IV line 30 which is itself coupled to the IV bag 32. The IV connector 28, line 30, and bag 32 are all well known in the art and are described here for illustrative purposes. IV fluid travels from the IV bag 32, through the IV line 30, and into the IV connector 28. There the fluid is transferred to the line 16 and ultimately into the introducer hub 14. The introducer cap 18 prevents any IV fluid from escaping the proximal end of the device 10 and directs it into the introducer sheath 12 and into the lumen 26 of the patient 24.

Because of the length of introducer line 16 and the tight seal that introducer cap 18 provides, none of the patient's blood or IV fluid may leak from the device 10 as the needle 20 is entering or exiting the introducer cap 18. This feature therefore eliminates the need to apply any outside pressure into the lumen 26 while an the introducer line 16 is being connected to an outside medical device, which also allows the operator to attach the IV connector 28 to the line 16 either before or after the introducer sheath 12 has been inserted into the patient 24.

In summary, the illustrated embodiment of the invention includes, but is not limited to, an apparatus for inserting a medical lead into the lumen of a patient comprising a introducer assembly including a side port; and a self-retracting needle assembly separate from the introducer assembly, wherein the needle assembly is wholly removable from the introducer assembly.

In one embodiment the introducer assembly comprises a central hub; a hollow sheath coupled to the distal end of the central hub; a cap coupled to the proximal end of the central hub; and a side port line coupled to and fluidicly communicated with the central hub and sheath.

In the illustrated embodiment the needle assembly comprises a housing; a moveable base disposed inside of the housing; a spring disposed between the moveable base and the distal end of the housing; and a needle coupled to the moveable base and axially disposed through the center of the spring.

The cap coupled to the distal end of the central hub is comprised of a material that allows it to be self-sealing each time it is punctured by an outside medical puncturing device, namely a needle coupled to the moveable base within the needle assembly. The side port line coupled to the central hub is in turn coupled to a supply device, which may be an intravenous bag.

The needle is long enough to transverse through the introducer assembly and puncture a lumen wall of the patient when distally extended from the housing. The sheath coupled to the distal end of the central hub comprises means for sliding distally over the needle after it has punctured a lumen wall of the patient.

The apparatus of claim 5 where the needle assembly comprises means for separating from the introducer assembly, which includes means for the needle from the introducer assembly and withdrawing entirely into the housing of the needle assembly.

The illustrated embodiment includes a method of inserting a sheath into a lumen of a patient comprising the steps of positioning a introducer assembly over a puncture site for the lumen of the patient where the sheath is to be inserted; disposing a needle from a needle assembly through the introducer assembly and into the patient; puncturing the lumen wall of the patient; sliding the distal portion of the sheath coupled to the distal end of the introducer assembly over the needle and into the lumen of the patient; withdrawing the needle from the lumen of the patient and introducer assembly completely into an enclosing housing without application of positive pressure to the sheath and at least in part by means of a self-retracting mechanism; and separating the needle assembly from the introducer assembly without blood loss from the introducer assembly.

The step of disposing a needle from a needle assembly through the introducer assembly comprises compressing a spring disposed in the needle assembly. Alternatively the step of disposing a needle from a needle assembly through the introducer assembly comprises puncturing a self-sealing cap disposed on the proximal end of the introducer assembly.

The step of withdrawing the needle from the lumen of the patient and the introducer assembly comprises retracting the needle into a housing of the needle assembly via the movement of an expanding spring disposed in the needle assembly.

The step of withdrawing the needle from the lumen of the patient and the introducer assembly comprises retracting the needle through a self-sealing cap disposed on the proximal end of the introducer assembly.

The step of separating the needle assembly from the introducer assembly comprises retracting the needle into a housing of the needle assembly via the movement of an expanding spring disposed in the needle assembly.

The step of separating the needle assembly from the introducer assembly comprises retracting the needle through a self-sealing cap disposed on the proximal end of the introducer assembly.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

I claim:

1. An apparatus for inserting a medical lead into the lumen of a patient comprising:
a introducer assembly including a side port; and
a self-retracting needle assembly separate from the introducer assembly,
wherein the needle assembly is wholly removable from the introducer assembly.

2. The apparatus of claim 1 where the introducer assembly comprises:
a central hub;
a hollow sheath coupled to the distal end of the central hub;
a cap coupled to the proximal end of the central hub; and
a side port line coupled to and fluidicly communicated with the central hub and sheath.

3. The apparatus of claim 2 where the needle assembly comprises:
a housing;
a moveable base disposed inside of the housing;
a spring disposed between the moveable base and the distal end of the housing; and
a needle coupled to the moveable base and axially disposed through the center of the spring.

4. The apparatus of claim 3 where the cap coupled to the distal end of the central hub is comprised of a material that allows it to be self-sealing each time it is punctured by a puncturing device.

5. The apparatus of claim 4 where the puncturing device is the needle coupled to the moveable base within the needle assembly.

6. The apparatus of claim 3 where the side port line coupled to the central hub is in turn coupled to a supply device.

7. The apparatus of claim 6 where the supply device is an intravenous bag.

8. The apparatus of claim 5 where the needle is long enough to transverse the introducer assembly and puncture a lumen wall of the patient when distally extended from the housing.

9. The apparatus of claim 8 where the sheath coupled to the distal end of the central hub comprises means for sliding distally over the needle after it has punctured a lumen wall of the patient.

10. The apparatus of claim 5 where the needle assembly comprises means for separating from the introducer assembly.

11. The apparatus of claim 10 where the means for separating from the introducer assembly comprises means for retracting the needle from the introducer assembly and withdrawing entirely into the housing of the needle assembly.

12. A method of inserting a sheath into a lumen of a patient comprising:
positioning a introducer assembly over a puncture site for the lumen of the patient where the sheath is to be inserted;
disposing a needle from a needle assembly through the introducer assembly and into the patient;
puncturing the lumen wall of the patient;
sliding the distal portion of the sheath coupled to the distal end of the introducer assembly over the needle and into the lumen of the patient;
withdrawing the needle from the lumen of the patient and introducer assembly completely into an enclosing housing without application of positive pressure to the sheath and at least in part by means of a self-retracting mechanism;
separating the needle assembly from the introducer assembly without blood loss from the introducer assembly.

13. The method of claim 12 where disposing a needle from a needle assembly through the introducer assembly further comprises compressing a spring disposed in the needle assembly.

14. The method of claim 12 where disposing a needle from a needle assembly through the introducer assembly further comprises puncturing a self-sealing cap disposed on the proximal end of the introducer assembly.

15. The method of claim 12 where withdrawing the needle from the lumen of the patient and the introducer assembly further comprises retracting the needle into a housing of the needle assembly via the movement of an expanding spring disposed in the needle assembly.

16. The method of claim 12 where withdrawing the needle from the lumen of the patient and the introducer assembly further comprises retracting the needle through a self-sealing cap disposed on the proximal end of the introducer assembly.

17. The method of claim 12 where separating the needle assembly from the introducer assembly further comprises retracting the needle into a housing of the needle assembly via the movement of an expanding spring disposed in the needle assembly.

18. The method of claim 12 where separating the needle assembly from the introducer assembly further comprises retracting the needle through a self-sealing cap disposed on the proximal end of the introducer assembly.
